(19) United States
(12) Patent Application Publication
(10) Pub. No.: US 2009/0093683 A1
(43) Pub. Date: Apr. 9, 2009

(54) SURGICAL PORTAL KIT FOR USE IN SINGLE INCISION SURGERY

(75) Inventors: Paul D. Richard, Newtown, CT (US); Gene A. Stellon, Burlington, CT (US); Steve L. Izzo, Naugatuck, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/194,831

(22) Filed: Aug. 20, 2008

Related U.S. Application Data
(60) Provisional application No. 60/998,046, filed on Oct. 5, 2007.

(51) Int. Cl. A61B 1/32

(52) U.S. Cl. 600/204

(57) ABSTRACT

A surgical kit for performing a surgical procedure includes at least two portal members, possibly, at least three portal members, and an obturator positionable within each of the portal members. Each portal member includes a portal housing and a portal sleeve extending from the portal housing, and having a passageway therethrough for reception of a surgical object, the portal head having a reduced profile, an object seal adapted to establish a fluid tight seal about the surgical object introduced therethrough and an insufflation port for permitting passage of insufflation gases. At least one of the at least two portal members includes an insufflation plug. The insufflation plug is positionable within the insufflation port to substantially close the insufflation port.

### CROSS-REFERENCE TO RELATED APPLICATION

The present application claims the benefit of and priority to U.S. Provisional Application Ser. No. 60/998,046, filed Oct. 5, 2007, the entire content of which being incorporated herein by reference.

### BACKGROUND

1. Technical Field

The present disclosure relates to a surgical portal kit for use in a surgical procedure. More particularly, the present disclosure relates to a surgical portal kit including at least one access port with reduced dimensional features to enable positioning of multiple ports within a single incision.

2. Background of the Related Art

Today, many surgical procedures are performed through small incisions in the skin, as compared to the larger incisions typically required in traditional procedures, in an effort to reduce both trauma to the patient and recovery time. Generally, such procedures are referred to as “endoscopic”, unless performed on the patient's abdomen, in which case the procedure is referred to as “laparoscopic”. Throughout the present disclosure, the term “minimally invasive” should be understood to encompass both endoscopic and laparoscopic procedures.

During a typical minimally invasive procedure, surgical objects, such as surgical access devices, e.g. trocar and cannula assemblies, or endoscopes, are inserted into the patient's body through the incision in tissue. In laparoscopic surgery, several cannulas may be positioned at different abdominal locations to access the peritoneal cavity to permit the introduction of the surgical instruments. Unfortunately, the presence of multiple cannulas within the operative area limits maneuverability about the patient thereby potentially impeding the surgical procedure. Furthermore, the creation of multiple incisions to accommodate the cannulas may increase trauma to the patient and recovery time.

### SUMMARY

Accordingly, the present disclosure is directed to a surgical kit for performing a surgical procedure, e.g., a laparoscopic surgical procedure, through a single incision in the skin. The surgical kit includes at least two portal members, possibly, at least three portal members, and an obturator positionable within each of the portal members. Each portal member includes a portal housing and a portal sleeve extending from the portal housing, and having a passageway therethrough for reception of a surgical object, an object seal adapted to establish a fluid tight seal about the surgical object introduced therethrough and an insufflation port for permitting passage of insufflation gases. The portal head has a reduced profile. At least one of the at least two portal members includes an insufflation plug. The insufflation plug is positionable within the insufflation port to substantially close the insufflation port.

The surgical kit may include insufflation tubing. The insufflation tubing is adapted for mounting to the insufflation port of each of the at least two portal members and is connectable to a source of insufflation fluids. A clamp may be mountable on the insufflation tubing for selectively opening and closing a lumen of the insufflation tubing.

A zero closure valve may be mounted within each of the at least two portal members. The zero closure valve is adapted to close in the absence of a surgical object.

A method for performing a laparoscopic surgical procedure is disclosed. The method includes the steps of:

forming a single incision in skin tissue overlying the peritoneal cavity;

introducing at least two portal members through the single incision and advancing the at least two portal members through different openings of the peritonea fascia tissue to access the peritoneal cavity, and

performing surgical procedures through the at least two portal members.

The step of introducing may include introducing a third portal member through the single incision and advancing the third portal member through peritonea fascia tissue to access the peritoneal cavity.

### BRIEF DESCRIPTION OF THE DRAWINGS

Various embodiments of the present disclosure are described hereinbelow with references to the drawings, wherein:

FIG. 1 is a view of a surgical kit apparatus in accordance with the principles of the present disclosure illustrating at least two portal members, an insufflation tube and an obturator of the surgical kit;

FIG. 2 is a side cross-sectional view of the housing of the portal member; and

FIG. 3 is a flow chart illustrating use of the surgical kit in performing a laparoscopic surgical procedure; and

FIG. 4 is a view illustrating positioning of multiple portal members within a single incision in the skin fascia in accordance with the method of using the surgical kit.

### DETAILED DESCRIPTION OF THE EMBODIMENTS

The surgical kit of the present disclosure is intended for use in the performance of a minimally invasive surgical procedure. The surgical kit permits the introduction and manipulation of various types of instrumentation while maintaining a fluid tight interface about the instrumentation to prevent gas and/or fluid leakage from the established pneumoperitoneum so as to preserve the atmospheric integrity of a surgical procedure. Examples of instrumentation include clip appliers, graspers, dissectors, retractors, staplers, laser probes, photographic devices, endoscopes and laparoscopes, tubes, and the like. Such instruments will be collectively referred to herein as “instruments or instrumentation”.

In the drawings and in the description which follows, in which like reference numerals identify similar or identical elements, the term “proximal” or “trailing” will refer to the end of the apparatus which is closest to the clinician during use, while the term “distal” or “leading” will refer to the end which is furthest from the clinician.

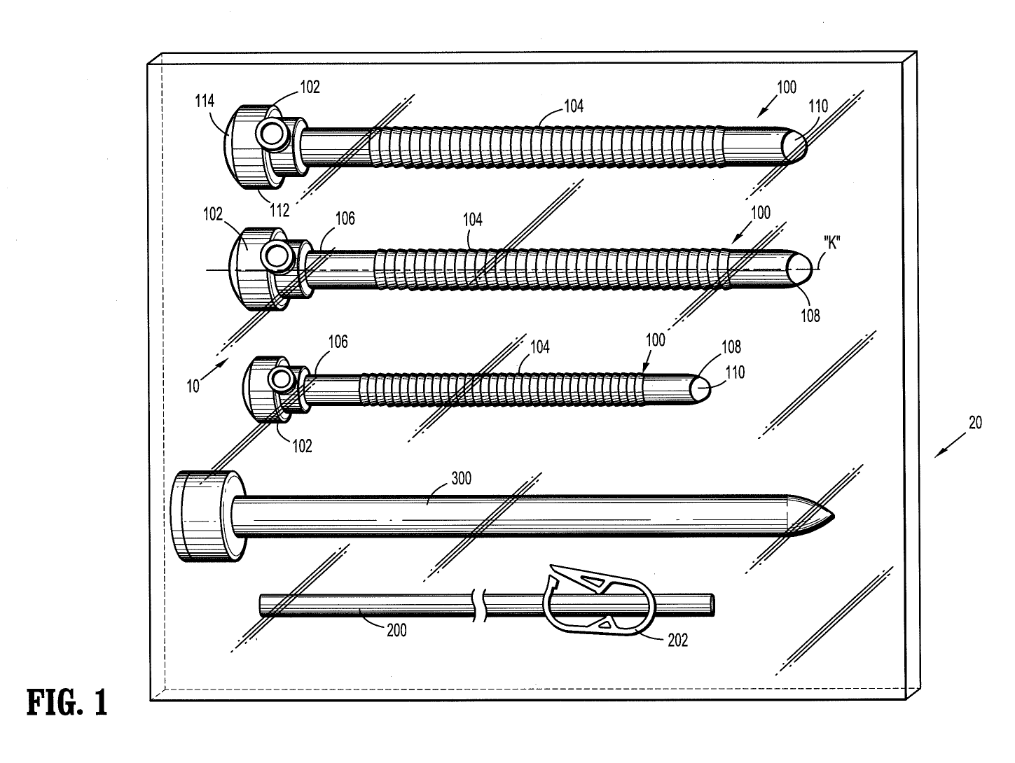

With reference to FIG. 1, a surgical kit 10 for use in a surgical procedure, e.g., a minimally invasive procedure is illustrated. Surgical kit 10 has particular application in laparoscopic surgery where the peritoneal cavity is insufflated to raise the cavity wall thereby providing access to tissue, organs etc within the cavity; however, uses in other minimally invasive procedures also are envisioned including arthroscopic, endoscopic or the like. Surgical kit 10 includes at least one, preferably, a plurality of portal members 100, an insufflation tubing 200 for connection to at least one of the portal members 100 and an obturator 300. The components of surgical kit 10 may vary in number or like depending on the surgical procedure to be performed. Various combinations of portal members 100, insufflation tubing and obturators are envisioned. Surgical kit 10 may be packaged within a tray 20 or the like and may be provided as a unit for a specific surgical procedure. For example, the surgical kit 10 may include portal members 100 of various diameters and/or lengths that are suitable for specific surgical procedures, e.g., hernia, bariatric, etc. for specific individuals, e.g., children, adults, etc. or for any other criteria.

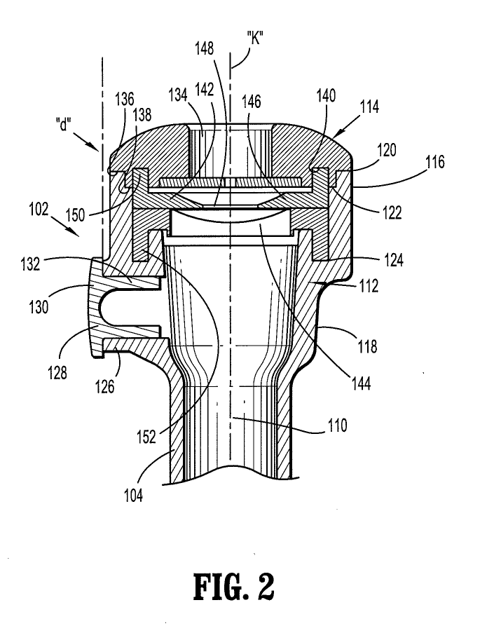

Referring now to FIGS. 1-2, each portal member 100 includes portal head or housing 102 and portal sleeve 104 connected to the housing 102. Portal sleeve 104 defines a longitudinal axis “k” extending along the length of the portal sleeve 104 and has proximal (or trailing) and distal (or leading) ends 106, 108. Portal sleeve 104 may be formed of any suitable medical grade material, such as stainless steel or other rigid materials, including polymeric materials, such as polycarbonate, or the like. Portal sleeve 104 may be transparent or opaque. The diameter of portal sleeve 104 may vary, but, typically ranges from about 3 millimeters (mm) to about 18 mm. In one embodiment, the diameter of portal sleeve 104 is about 5 mm.

Portal sleeve 104 may or may not include means for facilitating retention of the portal sleeve 104 within tissue. Such means may include a plurality of locking elements or ribs such as, e.g., the locking arrangement disclosed in commonly assigned U.S. patent application Ser. No. 11/170,824 to Smith filed Jun. 30, 2005, the entire contents of the '824 disclosure being hereby incorporated by reference herein. Portal sleeve 104 and portal head 102 further define internal longitudinal passage 110 extending through the portal sleeve 104 and the portal head 102 dimensioned to permit passage of surgical instrumentation.

Portal head 102 includes portal base 112 and portal cap 114 which is releasably mounted to the portal base 112. Any arrangement for mounting portal cap 114 to portal base 112 are envisioned including, but, not limited to, adhesives, cements, bayonet coupling, frictional fit, snap fit or the like. Portal head 102 defines first and second head segments 116, 118. First head segment 116 defines a substantially circular cross-sectional dimension transverse to the longitudinal axis “k”. In one embodiment, the maximum dimension or diameter of first head segment 116 ranges from about 5 millimeters (mm) to about 15 millimeters (mm), more preferably, about 8 millimeters (mm) to about 12 millimeters (mm). The maximum dimension or diameter of second head segment 118 ranges from about 3 millimeters (mm) to about 12 millimeters (mm), more preferably, about 5 millimeters (mm) to about 8 millimeters (mm). This dimensioning provides a substantially reduced profile to portal head 102 relative to conventional cannula assemblies thereby occupying substantially less space within the operative region above the operative site and facilitating the placement of multiple portal members 100 in adjacent sided by side relation within, e.g., a single incision, as will be discussed.

Portal base 112 defines outer peripheral shelf 120 extending orthogonal to longitudinal axis “k”, a second step or shelf 122 inward of the outer annular shelf 120 and annular mounting recess 124 which is disposed inward of the second shelf 122.

Portal base 112 further define insufflation port 126 which depends radially outwardly from second head segment 118. Insufflation port 126 permits the introduction and/or release of insufflation gases through longitudinal passage 110 of portal member 100. The disposition of insufflation port 126 adjacent second head segment 118 results in only a slight extension of the insufflation port 126 beyond the perimeter of first head segment 116. In particular, insufflation port 126 extends a distance “d” beyond first head segment 116. Distance “d” is substantially negligible ranging from about 1 millimeter (mm) to about 3 millimeter (mm) thereby also minimizing the profile of portal head 102 within the operative region and the potential of obstruction of the portal base 112 with activities, tasks performed during the surgical procedure. Insufflation port 126 may be supplied with insufflation plug 128 which is selectively positionable within the insufflation port 126. Insufflation plug 128 may be fabricated from a suitable polymeric, elastomeric or foam material and is intended to close the insufflation port 126 to prevent leakage of insufflation gases. Insufflation plug 128 defines flat plug head 130 and plug extension 132 which is received within insufflation port 126. Plug extension 132 is dimensioned to establish a sealing relation with the internal surface area of insufflation port 126.

Portal cap 114 defines central opening 134 having an internal dimension or diameter approximating the internal diameter of portal sleeve 104. The outer diameter of dimensioning of portal cap 114 generally approximates the outer diameter of portal base 112 as shown. Portal cap 114 defines outer peripheral shelf 136, second shelf 138 disposed radially inward of the peripheral shelf 136 and annular mounting recess 140 which is inward of the second shelf 138. Outer peripheral shelf 136 and second shelf 138 of portal cap 114 reside on respective outer peripheral shelf 120 and second shelf 122 of portal base 112 when in the assembled condition of the components. Portal cap 114 and portal base 112 may be adhered along respective shelves to secure the two components to each other.

Portal head 102 includes object seal 142 and zero closure valve 144. Object seal 142 may be any seal adapted to form or establish a sealing relation with a surgical instrumentation introduced through portal member 100. In one embodiment, object seal 142 is a septum seal defining inner seal segment 146 having central aperture 148. Inner seal segment 146 defines a cross-sectional dimension or thickness which gradually decreases toward central aperture 148 and longitudinal axis “k”. Object seal 142 may be fabricated from a suitable elastomeric material, gel material, foam material or a fluid filled cavity, having sufficient compliance to form a seal about the surgical instrumentation. Object seal 142 preferably comprises a resilient material in at least the region of inner seal segment 146 to form a substantial seal about an instrument inserted through central aperture 148. Object seal 142 may be monolithically formed or composed of several components interconnected to each other. In one embodiment, object seal 142 includes a resilient elastomer (e.g., polyisoprene or natural rubber) and has a layer of fabric impregnated on each surface of the resilient seal. The fabric may be of any suitable fabric for example, a SPANDEX material containing about 20% LYCRA and about 80% NYLON available from Milliken. A suitable object seal is disclosed in commonly assigned U.S. Pat. No. 6,702,787 to Racenet et al. and/or U.S. Pat. No. 6,482,181 to Racenet et al., the entire contents of each disclosure being incorporated herein by reference.

Object seal 142 includes peripheral flange 150 extending in a proximal or trailing direction. Flange 150 is dimensioned to be received within annular mounting recess 140 of portal cap 114 to facilitate securement of object seal 142 within portal head 102.

Zero closure valve 144 is mounted adjacent object seal 142 and may be in contacting relation with the object seal 142. Zero closure valve 144 may be any valve adapted to close in the absence of the surgical object and/or in response to the pressurized environment of the underlying insufflated body cavity. Zero closure valve 144 may be a duck bill valve, trumpet valve, gel seal, foam seal, bladder seal or the like. In one embodiment, zero closure valve 144 includes outer peripheral flange 152 depending in a leading or distal direction. Flange 152 is received within corresponding annular 124 recess of portal base 112 to facilitate securement of the zero closure valve 144 within portal head 102.

Portal head 102 is assembled by positioning zero closure valve 144 adjacent portal base 112 with peripheral flange 152 being received within annular mounting recess 124 of the portal base 112. Zero closure valve 144 is placed in, e.g., superposed relation, with object seal 142. Portal cap 114 is positioned on portal base 112 with peripheral flange 150 of object seal 142 being received within annular mounting recess 140 of portal cap 114. Portal cap 114 is then secured relative to portal base 112 by any of the aforementioned means including, e.g., adhering the portal cap 114 and the portal base 112 along respective shelves.

Surgical kit 10 may incorporate portal members 100 identical in size and type, or alternatively, having different sizes, lengths, diameters etc.

Referring again to FIG. 1, insufflation tubing 200 of surgical kit 10 is adapted for introduction within insufflation port 126 of portal base 112 and establishes a frictional fit with the interior wall of the insufflation port 126 to releasably secure the tubing 200 to portal head 102. Other means for securing insufflation tubing 200 to portal head 102 are envisioned including bayonet coupling, snap fit or the like. Insufflation tubing 200 is connectable to a source of insufflation gases. Insufflation tubing 200 may further include tube clamp 202. Tube clamp 202 is adapted to open and close to selectively open or close the lumen of insufflation tubing 200. Any conventional tube or catheter clamp may be utilized.

Referring still to FIG. 1, obturator 300 may be blunt, a non-bladed, or a sharp pointed instrument positionable within the passageway of the portal member 100. Obturator 300 is utilized to penetrate the peritoneal wall to introduce portal member 100 through the peritoneal wall, and then subsequently is removed from the portal member 100 to permit introduction of the surgical instrumentation utilized to perform the procedure through the longitudinal passage 110 of the portal member 100.

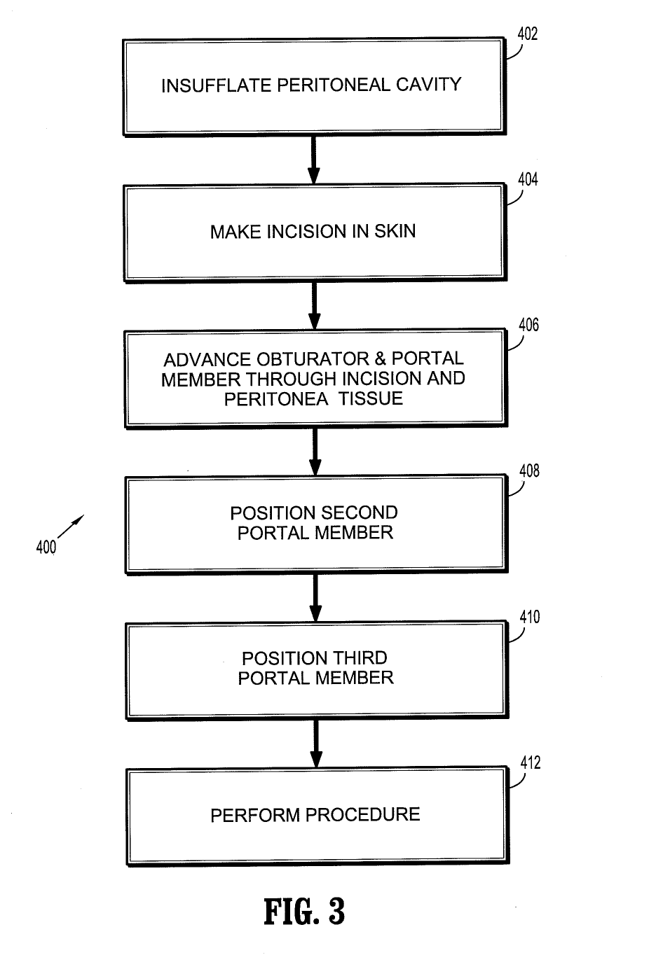

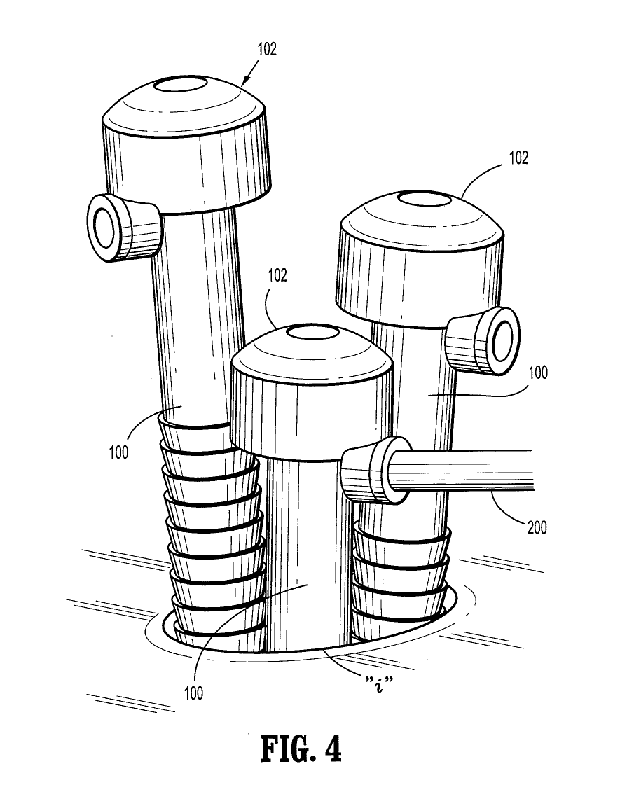

Referring now to the flow chart of FIG. 3, the use and function of surgical kit 10 will be discussed during the course of a laparoscopic minimally invasive procedure (STEP 400). Initially, the peritoneal cavity is insufflated with a suitable biocompatible gas such as, e.g., CO₂ gas, such that the cavity wall is raised and lifted away from the internal organs and tissue to provide access the organs. (STEP 402) The insufflation may be performed with an insufflation needle or similar device, as is conventional in the art. Either prior or subsequent to insufflation, a single incision is made in at least the skin fascia, the dimensions of which may be varied dependent upon the nature of the procedure. (STEP 404) Thereafter, obturator 300 is mounted within portal member 100 and the assembled unit is positioned within the incision within the skin. Obturator 300 is manipulated through the skin incision to penetrate through deep fascia or peritonea tissue, e.g., the peritoneal lining, to access the underlying peritoneal cavity. (STEP 406) Thereafter, additional portal members 100 from the surgical kit 10 may be used with the obturator to access the peritoneal cavity in a similar manner. In one method of operation, a second portal member 100 is introduced within the previously formed skin incision and advanced in conjunction with the obturator to create another opening in the peritonea fascia to access the peritoneal cavity (STEP 408). This procedure thereby positions second portal member 100 in adjacent relation to first portal member 100. A third portal member 100 optionally may be positioned within the same skin incision and advanced with the obturator to define another opening within the deep peritonea fascia tissue. (STEP 410) FIG. 4 illustrates three portal members 100 accessing the peritoneal cavity. The respective low profile dimensioning of portal members 100, particularly, portal heads 102 enables such placement of portal members 100 in side by side relation. Portal members 100 may be positioned at different depths with respect to the each other to laterally displace respective portal heads 102 of the portal members 100 to maximize available spacing within the operative region as depicted in FIG. 4. One or more portal members 100 may have insufflation tubing 200 mounted to its insufflation port 126 to selectively supply insufflation gases to the peritoneal cavity. The remaining portal members 100 may have insufflation plugs 128 mounted within their respective insufflation ports 126 to prevent escape of insufflation gases through the insufflation ports 126. Thereafter, surgery is performed with instrumentation positioned within any of portal members 100 accessing the peritoneal cavity. (STEP 412)

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

What is claimed is:

1. A surgical kit for performing a surgical procedure, which comprises:
at least two portal members, each portal member including:
a portal housing and a portal sleeve extending from the portal housing, and having a passageway therethrough for reception of a surgical object, the portal head having a reduced profile;
an object seal adapted to establish a fluid tight seal about the surgical object introduced therethrough; and
an insufflation port for permitting passage of insufflation gases; and
an obturator positionable within each of the at least two portal members.

2. The surgical kit according to claim 1 wherein at least one of the at least two portal members includes an insufflation plug, the insufflation plug positionable within the insufflation port to substantially close the insufflation port.

3. The surgical kit according to claim 2 including insufflation tubing, the insufflation tubing adapted for mounting to the insufflation port of each of the at least two portal members and connectable to a source of insufflation fluids.

4. The surgical kit according to claim 3 including a clamp mountable on the insufflation tubing for selectively opening and closing a lumen of the insufflation tubing.

5. The surgical kit according to claim 1 wherein including a zero closure valve mounted within each of the at least two portal members, the zero closure valve adapted to close in the absence of a surgical object.

6. The surgical kit according to claim 1 including at least three portal members.

7. The surgical kit according to claim 1 wherein the portal sleeves of the at least two portal members define different internal dimensions.

8. A method for performing a laparoscopic surgical procedure, comprising the steps of:
forming a single incision in skin tissue overlying the peritoneal cavity;
introducing at least two portal members through the single incision and advancing the at least two portal members through different openings of the peritonea fascia tissue to access the peritoneal cavity; and
performing surgical procedures through the at least two portal members.

9. The method according to claim 8 wherein the step of introducing includes introducing a third portal member through the single incision and advancing the third portal member through peritonea fascia tissue to access the peritoneal cavity.
